(19) United States
(12) Patent Application Publication
(10) Pub. No.: US 2001/0047010 A1
(43) Pub. Date: Nov. 29, 2001

(54) Compositions and methods for treating particular chemical addictions and mental illnesses

(76) Inventor: Jose Pozuelo, Barcelona (ES)

Correspondence Address: Richard J. Minnich, Esq., FAY, SHARPE, FAGAN, MINNICH & McKEE, LLP, 1100 Superior Avenue, Suite 700, Cleveland, OH, 44114, US

(21) Appl. No.: 09/773,336

(22) Filed: Jan. 31, 2001

Related U.S. Application Data
(63) Continuation-in-part of application No. 09/073,337, filed on May 5, 1998, now abandoned.

(51) Int. Cl. A61K 31/47; A61K 31/195; A61K 31/45

(52) U.S. Cl. 514/310; 514/327; 514/567

(57) ABSTRACT

Pharmaceutical compositions and related methods are disclosed for treating addiction to an array of agents such as heroin, narcotics, cocaine, amphetamines and/or marijuana. Compositions and methods are also disclosed for treating alcoholism and dependence on nicotine intake, such as smoking. Also disclosed are pharmaceutical compositions and related methods for treating various mental illnesses or conditions, such as for example, schizophrenia and manic depressive psychosis.

### CROSS REFERENCES TO RELATED APPLICATIONS

This is a continuation-in-part of U.S. application Ser. No. 09/073,337 filed May 5, 1998.

### BACKGROUND OF THE INVENTION

1. Field of the Invention

The present invention relates to compositions, namely pharmaceutical compositions, for treating patients who are addicted to agents such as narcotics, cocaine, amphetamines, alcohol and/or marijuana. The present invention compositions are also utilized for treating tobacco addiction, such as for example in the form of smoking of cigarettes or cigars, or any addictive condition involving the intake of nicotine. The present invention also provides related methods for administering such compositions and treating such addictions. Furthermore, the present invention compositions and methods are effective for treating schizophrenia and manic depressive illnesses. Treatment of such manic depressive illnesses in accordance with the present invention, results in total cessation of the acute hallucinatory or delusional symptoms of the manic phase after treatment is initiated. Moreover, such treatment prevents the development of the ensuing depressive phase.

2. Description of the Related Art

In all the noted addictions, there are alterations in the synthesis, release, and/or re-uptake of the neurotransmitter dopamine. In schizophrenia and the manic phase of manic-depressive psychosis (typically referred to as bipolar illness), alteration of dopamine or mechanisms involving dopamine may occur. In view of the significant role dopamine plays, neuroleptics, and all dopamine receptor blockers have been used in the treatment of conditions (schizophrenia and mania) for many years. However, treatment regimens utilizing typical neuroleptics, such as Eskazine (Trifluoperzine), Meleril (Thioridazine), or Orap (Pimozide), require many days or weeks of continuous treatment in order to control the acute symptoms of such conditions. And, all currently known techniques for treating schizophrenia and manic depressive illnesses have met limited success. Moreover, all currently known approaches for treating chemical addictions involving narcotics, cocaine, amphetamines, alcohol, marijuana, and nicotine have limited success.

It would be highly desirable to decrease treatment time periods for such addictions. Furthermore, there is a need for an improved approach for treating these addictions and mental conditions. Accordingly, there is a need for a composition and method that provides improved success and response for treating these noted addictions and illnesses.

### SUMMARY OF THE INVENTION

The present invention achieves all of the foregoing objectives and provides, in a first aspect, a composition comprising an effective amount of alpha-methyl-para-tyrosine (AMPT) or closely related compounds in combination with an effective amount of Haloperidol (Haldol). In another aspect, the present invention provides a method for treating addiction to heroin, narcotics, alcohol, marijuana, and/or other agents by administering an effective amount of alpha-:methyl-para-tyrosine in combination with an effective amount of Naltrexone. In yet another aspect, the present invention provides a method for treating schizophrenia or manic depressive psychosis by administering an effective amount of alpha-methyl-para-tyrosine in combination with an effective amount of Haloperidol.

### DESCRIPTION OF THE PREFERRED EMBODIMENTS

The present invention comprises the use of two potent neuroleptics AMPT (alpha-methyl-para-tyrosine) and Haldol (Haloperidol), the combination of which has surprisingly been found to be effective for treating addictions to heroin, narcotics, cocaine, amphetamines, alcohol and nicotine, marijuana and mental illnesses such as schizophrenia and manic depressive psychosis. Alpha-methyl-para-tyrosine (C₁₀H₁₃NO₃), has the following structural formula.

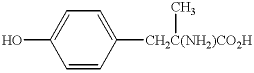

Alpha-methyl-para-tyrosine, or AMPT as typically referred to herein, is commercially available from an array of sources.

Haloperidol is 4-[4-(p-chlorophenyl)-4-hydroxy-piperidino]-4′-fluorobutyrophenone. Haloperidol is the first of the butyrophenone series of major tranquilizers. Haloperidol has the following structural formula.

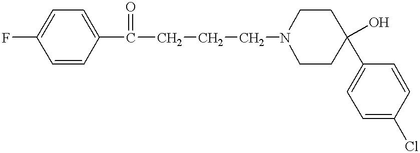

Haloperidol is available from McNeil Pharmaceutical under the designation HALDOL.

The present invention also comprises the use of an alkalinizer to adjust urine pH to about 8, and at least above about 7.4. Such adjustment enables the administration of AMPT in therapeutic amounts, high enough to prevent withdrawal symptoms, to abolish craving of the addictive agents, and to reverse the pathological symptoms (e.g. hallucinations and delusions) in the noted mental illnesses, without the production of AMPT crystalluria. The preferred embodiment alkalinizer is Polycitra, manufactured by Willen Drug Company. Polycitra contains 30 grains of citric acid, 45 grains of sodium citrate and 50 grains of potassium citrate for every 30 ml of the syrup base solution. Polycitra is also available from Baker Norton Pharmaceuticals, Inc. in liquid syrup forms. These forms comprise, per teaspoon of 5 ml, 550 mg potassium citrate, 500 mg sodium citrate dihydrate, and 334 mg citric acid monohydrate. It will be understood that the present invention includes other agents for rendering urine alkaline, such as for example sodium bicarbonate and ammonium chloride, but we found Polycitra to be effective and the most palatable.

The therapeutic doses used for administering the combination of AMPT and Haloperidol depend upon the condition to be treated and patient-related factors. The dosage may also vary depending on the chronicity and degree of tolerance of the addictions and on the intensity and quality of the florid symptoms in the cases of schizophrenia and manic-depressive psychosis. Therefore, the dosage level for each of AMPT and Haloperidol can only be determined empirically. However, as a general rule, higher doses of AMPT are required for chronic cases of addiction with high tolerance and for chronic and florid symptoms associated with the noted mental illnesses. Dosages of AMPT typically range between about 1 to about 200 mg per kilogram of body weight per day upon initiation of the treatment. Preferred dosages of AMPT generally range from about 15 mg to about 50 mg per kilogram of body weight per day during early phases of treatment. It will be appreciated that as treatment continues, these dosage levels may be reduced in accordance with patient response. Initial dosages of Haloperidol generally range from about 0.015 to about 1.0 mg per kilogram of body weight per day. Preferred dosages of Haloperidol generally range from about 0.05 mg to about 0.06 mg per kilogram of body weight per day. Dosage levels may be reduced as treatment progresses. With regard to all of the noted dosages, the total daily dosage level is usually given over several administrations over the course of a day, such as 2, 3 or 4 times. These dosages are generally referred to herein as “an effective dosage amount.” It will be understood that the present invention includes dosages greater or lesser than these amounts.

The invention also includes the use of Naltrexone, a well known narcotic antagonist that the present inventor has found useful for preventing the relapse of narcotic addicts, already treated and free of narcotics. In accordance with the present invention, Naltrexone was also discovered to be effective for preventing relapse into alcohol and marijuana from initiation of treatment and continued for at least three months. That is, Naltrexone may be administered for treating alcoholism and marijuana even after the administration of AMPT and Haloperidol has been discontinued. It has also been discovered that when treating alcoholics, low doses of Haloperidol help to abolish craving and its effects in a shorter time period than when only AMPT is used. This treatment is illustrated in several case reports herein. It is remarkable that none of the Naltrexone maintained patients, described in case studies below, relapsed into alcohol during the following two years. The maintenance dose of Naltrexone for a typical patient, is generally about 50 mg daily for approximately 3 months, and on alternate days for another 3 additional months.

Polycitra or nearly any urine alkalinizer is administered concurrently with AMPT, particularly during portions of treatment in which AMPT is administered in relatively high doses, for example, Polycitra is administered in an amount of about 15 ml of the syrup, 3-4 times a day in order to maintain a pH above 7.4. The exact amount of the urine alkalinizer required for proper alkalinization may vary from day to day, according to the diet received. It is very important that urine pH be checked every morning, noon, and late evening to ensure that the pH is always above 7.4, preferably greater than about 7.8, and most preferably about 8.0. At a minimum, the urine alkalinizer should be administered in amounts such that urine pH does not fall below 7.4.

Long term administration of AMPT is generally not necessary. Long term administration of AMPT is required chronically only for the treatment of schizophrenia and mania, while in all other conditions noted herein, only a temporary treatment, generally not exceeding 6-8 weeks, is required except for cocaine where AMPT low dosage is maintained for about one year. However, the present invention includes longer or shorter treatment periods.

The present invention is directed to the administration of ANPT in combination with Haloperidol. It is to be understood that the present invention does not require the concurrent or simultaneous administration of these agents, but instead, treatment regimens involving those agents. That is, the invention encompasses treatment methods in which on the same day AMPT is first administered with or without administration of Haloperidol, depending on the case. It is important, however, that the administration of one agent occur immediately before or during the effect of the other agent. These practices are described in greater detail in the case studies set forth below, in addition to the use of Naltrexone.

The components of the present invention composition, i.e. AMPT, Haloperidol, and the agent for rendering urine alkaline, can be mixed or otherwise administered in combination with other materials. For example, in the case of a tablet, the composition can also include fillers, binders, and diluents such as lactose, methylcellulose, talc, gum tragacanth, gum acacia, agar, polyvinylpyrrolidone, calcium stearate, and/or corn starch. In the case of a liquid solution or suspension for oral administration, the composition can include a filler such as sodium carboxymethylcellulose and/or syrup, e.g., a glycerine based syrup. In the case of a parenteral solution or suspension, the composition may comprise a suitable solvent or other liquid such as a physiologic solution.

The present invention composition and treatment regimen was utilized by treating patients, all with well documented histories of addiction and dependence on various narcotics, cocaine, amphetamines, cigarettes, alcohol, marijuana, and/or schizophrenia or manic depressive illnesses. All of these patients submitted voluntarily to the study. All patients had severe conditions and would not respond or failed to respond to other available types of treatment.

The present invention is particularly well suited for treating the following states or addictions: (i) addiction to heroin, narcotics, cocaine, and amphetamines, and marijuana (ii) addiction to alcohol; (iii) schizophrenia or mania; and (iv) addiction to nicotine or smoking in general. The general approach for treating each of these states is described below.

### Use of AMPT, a Urine Alkalinizer, and Haloperidol for Treating Addiction to Heroin, Narcotics Cocaiine, and/or Amphetamines

Two major aspects of the treatment of a drug addict relate to abolishing the craving and dependence, be it psychological or physical, and to the prevention of the withdrawal or abstinence syndrome.

In order to abolish craving and prevent withdrawal symptoms, an almost universal method has been used, consisting of replacement of the offending drug with one or more acceptable, although still addictive, drugs. A more desirable method, and in accordance with the present invention, is the use of a compound, combination of compounds, or composition that alters the biochemical mechanism of addiction and abolishes the craving and withdrawal symptoms.

Initial experimental work with morphine addicted monkeys demonstrated that treatment with AMPT abolishes the craving for morphine and the manifestations of the abstinence syndrome. When the results of these investigations were first made known, it was suggested that AMPT could be used for the treatment of narcotic and amphetamine addiction and other illnesses where the catecholamines, dopamine among them, were playing a fundamental role in the promotion of the addictive states.

The promising results of the previously noted experiments with monkeys led to the trial of AMPT for patients addicted to heroin. Unfortunately, all patients developed AMPT crystalluria, as in retrospect had also occurred in the monkeys. As a consequence, treatment with AMPT was discontinued.

AMPT affects the enzyme tyrosine-hydroxilase (TH) which regulates the synthesis of dopamine and norepinephrine, and therefore is responsible for the amount of their by-products. These by-products create a feedback mechanism that influences the activity of the enzyme tyrosine-hydroxilase.

Once known that AMPT produced crystalluria, which prevented its use in humans, the present inventor conducted research to find a solution to the crystalluria problem. Specifically, this research was directed to provide a composition containing AMPT that could be used in the treatment of patients suffering from different conditions and without the formation of AMPT crystalluria. This was accomplished with further research in animals, such as mice, rats, mongrel and beagle dogs, to which AMPT with a urine alkalinizer was administered. The alkalinization was obtained with Polycitra, administered orally, and in an amount to obtain a urine pH of about 8.

Upon demonstration of the safety of AMPT if administered with an alkalinizer, such safety confirmed by autopsy, macroscopic and microscopic, including electron-microscopy, and studies of different organs and tissues of animals treated with AMPT and a urine alkalinizer, the present inventor advanced its use to humans.

Patients addicted to heroin and narcotics in general, cocaine and/or amphetamines, quite frequently polydependent, responded to the AMPT, administered with a urine alkalinizer, with cessation of the craving and without manifestations of withdrawal. The narcotic dependent patients were transferred to MST (oral morphine), from the irregular doses of heroin or other substitutes they might otherwise take, in order to satisfy their craving and prevent manifestation of abstinence during the evaluation. A period of 24 hours, during which a physical examination and basic analytic tests were performed, was used for each patient in order to verify their condition and also to stabilize urinary pH and monitor vital signs. A period of 3-4 days was utilized before initiating treatment with AMPT. During this period, studies on catecholamine levels before and after treatment were conducted. During the first initial week, patients treated with AMPT were carefully monitored for vital signs. However, as we gained experience, it became obvious that many patients could have been treated ambulatorily. For the addiction cases, after stabilizing the dose of MST that the patient needed, the administration of AMPT was initiated at an average dose of 115 mg per kilogram of body weight, per day, adjusting it every two days according to the response of the patient. The required doses of the alkalanizer Polycitra had been previously established in all patients prior to the administration of AMPT. When the dosage of AMPT administered orally reached 80 mg, per kg of body weight, per day, the morphine was discontinued and given only upon request by the patient. Naltrexone was also administered concomitantly with AMPT, from the initiation of the treatment on alcoholics, and on narcotic addicts when free of narcotics on their metabolites.

Concurrently, with administration of AMPT, all patients were started on Haloperidol, at a dose of 10 mg t.i.d. (total intake per day). The doses were adjusted in each case in order to reduce the degree of somnolence, according to the criteria decided for each patient. The Haloperidol dose was reduced to about 60% after 2 days and on the following week to an average of 3 mg per day, to be discontinued 3 days later.

Administration of AMPT, in low dosage, and Naltrexone was continued for at least 6 months in all narcotic addicts and alcoholics. For cocaine addicts, after initial AMPT treatment, administration of AMPT was maintained for at least a year so that the craving for the drug could remain suppressed. For cases of amphetamine addiction, AMPT was continued for at least a year as, similarly to cocaine addiction, no antagonists exist. AMPT maintenance for the treatment of amphetamines required dosages of about 40% less than the dosages required for treatment of narcotics or cocaine.

Amphetamine addicted patients were maintained, while undergoing initial evaluation, on a regular dosage level of amphetamines, 60% lower than the estimated dose that the patients had been taking before treatment. The amphetamines were discontinued after reaching a dosage level of 60 mg of AMPT per kg of body weight per day, but the patients being told that amphetamines would be given if they still craved them.

In all cases, urine specimens were checked 3 times daily for patients receiving moderate to high dosages of AMPT, to determine urinary pH and to look for crystals of AMPT in the sediment. Also, in all patients treated, an intake of fluids above 2 liters per day was recommended to force diuresis and further prevent and minimize the formation of AMPT crystals in the urine.

The present inventor previously described treatment of patients addicted to narcotics and amphetamines by administering AMPT and a urine alkalinizer in U.S. Pat. No. 4,117,161, herein incorporated by reference. Although satisfactory in many respects, there remains a need for an improved treatment regimen and/or composition(s) or agent(s) for treating the addictions and mental disorders described herein. As previously noted, the treatment of addiction to narcotics and amphetamines by administering AMPT is known. The present invention provides a significant improvement over all known treatment regimes, particularly those based upon the sole administration of AMPT.

Set forth below in Table 1 is a summary of treatment duration for patients with dual disorder, i.e. bipolar illness. Table 1 lists treatment schedules based upon administering AMPT alone, as compared to treatment schedules based upon administering the combination of AMPT and Haloperidol. The surprising and unexpected advantages of the present invention are clearly evident. The average time period of hospitalization decreased from 47 days to only 12 days. This remarkable decrease in length of treatment results in significant cost savings.

TABLE 1
PATIENTS TREATED

| | | |
|---|---|---|
| A. | Old Method (AMPT) | |
| | Number of Patients | 425 |
| | Patients with Dual Disorder | 335 (79.8%) |
| | Average Length of Hospitalization | 47 days |
| | Shortest Hospitalization | 26 days |
| | Longest Hospitalization | 97 days |
| B. | New Method (AMPT and HALOPERIDOL) | |
| | Number of Patients | 137 |
| | Patients with Dual Disorder | 114 (83.2%) |
| | Average Length of Hospitalization | 12 days |
| | Shortest Hospitalization | 5 days |
| | Longest Hospitalization | 21 days |

The present inventor discovered the striking advantages and benefits in utilizing the combination of AMPT and Haloperidol while studying the biochemistry of Manic-Depressive-Psychosis (MDP), also referred to as bipolar illness.

While studying human subjects, diagnosed with bipolar illness, the effects of administering Haloperidol on the metabolites of the neurotransmitter dopamine (DA) were analyzed. It was noted that the concentrations in urine, of homovanilic acid (HVA), a dopamine metabolite, were increasing during Haloperidol administration. The concentration of HVA reached a maximum level in 3-4 weeks, after which, the HVA concentration started to decrease, even if Haloperidol was continued to be administered.

The present inventor concluded that:

a) The administration of Haloperidol produced an increased release of dopamine, which results from an increase of dopamine synthesis in the pre-synaptic terminal.

b) The increase of dopamine synthesis and release stops after 3-4 weeks of Haloperidol administration. This is probably a consequence of an exhaustion of the dopamine mechanisms to maintain the replenishment of the dopamine vesicles in the pre-synaptic terminal.

c) The mechanism involves a similarity with general endocrine glands, where the pre-synaptic synthesis of the corresponding neurotransmitter increases in order to overcome any obstacle in the post-synaptic receptor.

d) The obstacle created by the noted administration of Haloperidol, would be the blockage of the post-synaptic dopamine receptor (D2) by the drug.

The present inventor did not rule out the possibility that, as a consequence of the above noted findings and proposed mechanisms, it would be possible, perhaps, to normalize the period of increased synthesis and release of dopamine, without waiting for the supposed period of time needed to exhaust the dopamine replenishment of the pre-synaptic terminal vesicles. The present inventor believed that it could be accomplished by the simultaneous administration of Haloperidol and alpha-methyl-para-tyrosine (AMPT). The Haloperidol would block the D2 receptor immediately, prompting the increase of dopamine synthesis, that would be decreased by the well known action of AMPT, by inhibiting the enzyme tyrosine hydroxilare (TH), considered to be “the pace-maker” in the synthesis of catecholamines.

Furthermore, the present inventor believes that AMPT not only inhibits TH when dopamine synthesis is increased, but also increases TH activity when dopamine synthesis is decreased, therefore regulating the production of dopamine.

The confirmation of this hypothesis in clinical practice did not take very long. Patients with maniac, paranoid, schizophrenia, and dependence of different types of addicting drugs, could be treated by combining the administration of Haloperidol and AMPT in an incredible shorter time period than the time needed when only AMPT is utilized. Furthermore, by treating the addictive conditions in only a few days, it was possible to start treatment of the commonly associated psychiatric conditions—up to 83% of patients, almost immediately. Most recently, treatment of both conditions, at the same time, has been conducted.

Set forth below are detailed descriptions of particular treatment regimens for certain types of addictions and disorders. Case reports 16-19 provide a comparison of treatment with only AMPT and treatment with AMPT in combination with Haloperidol. Case reports 14 detail the treatment for addiction in accordance with the present invention. The other case reports detail treatment regimes for other addictions and disorders in accordance with the present invention.

### Use of AMPT, with a Urine Alkalinizer, Naltrexone and Haloperidol for Treating Alcoholism

Alcohol is an addictive substance, known for many centuries as causing a condition called alcoholism, that can have many different manifestations and consequences.

While conducting previous research, the present inventor found that laboratory animals (rats) drinking a 25% solution of alcohol during 4 months, responded, when tested with Naltrexone, with similar acute withdrawal symptoms as rats having received increased doses of methadone solution in their drinking water. Subsequently, it was observed that rats made dependent on alcohol (25% alcohol solution) would prefer again to drink plain water, instead of the alcohol solution upon which they had been made dependent, when treated with AMPT (300 mg per kg of body weight), with their preference being manifested after 24 days of treatment. In contrast, alcoholic rats that did not receive AMPT preferred to continue drinking an alcohol-water solution.

The previously demonstrated cross tolerance with methadone and the response of animals to the narcotic antagonist Naltrexone, led the present inventor to approach the treatment of alcoholics with AMPT, Naltrexone and Haloperidol. The treatment regimen was similar to the regimen successfully used with heroin in narcotic addicted patients, and with same or similar positive results. However, one significant difference with regard to narcotics is that alcohol is a weaker addictive substance than heroin and so imparts a lesser degree of disability. One year after treatment of alcoholic patients, the success of the present invention composition and methodology was 100%, in terms of no relapse to drinking alcohol. It has also been found that for treating alcoholism, in some instances, only AMPT and Naltrexone are necessary. However, by adding Haloperidol, the effectiveness of the AMPT is enhanced and the positive effects appear sooner. The case reports of 5-7 below, illustrate in greater detail this aspect of the present invention.

### Use of AMPT, with a Urine Alkalanizer and Haloperidol to Treat Marijuana Dependence

No drug has raised more debates and has had more controversy than the smoking of marijuana or its more concentrate derivative, hashish. In the opinion of this inventor marijuana or its most psycho-active by-products; tetra-hydro-cannabinoids (THC), have an addictive potential in humans that is directly proportional to the amount ingested, generally by smoking, the activity of the compound and the period of time over which it has been consumed. Cannabis psychosis is well known by arm psychiatrists serving legionary soldiers having their headquarters in northwest Africa, where it was customary to allow the soldiers to smoke “grifa” (a marijuana variety) the day before battle, producing euphoria and removing fear on the part of “aguerridos” legionaries, who entered battles without being afraid of bullets.

### Use of AMPT, with a Urine Alkalinzer, and Haloperidol, for Treating Schizophrenia and Mania

In schizophrenic patients there are two major types of symptoms: (i) a disturbance of mood and a disorganization of the thinking process and (ii) hallucinations or delusions. Both aspects render the individual incapable of dealing with the requirements of everyday living.

Schizophrenia has long been suspected as caused by one or more biochemical factors. The identification of the biochemical factors, whether genetically induced or triggered by environmental situations, enzymatic, electrolytic in nature, etc., has been the object of many different investigations. Even if the etiologic agent of all these disturbances is unknown, an abnormality in the mechanism of the neurotransmitters is evidently involved. It is well known that the catecholamines, specifically dopamine and noradrenaline, are two fundamental neurotransmitters. It has been speculated that alterations in the synthesis, release, catabolism, or re-uptake of these compounds could be responsible for the symptoms of schizophrenia, where dopamine has been considered the main neurotransmitter involved, according to reports of most leading researchers. In order to manipulate the mechanism of action of neurotransmitters, different therapeutic agents have been used, namely the neuroleptics such as Chlorpromazine, Thioridazine, Trifluoperazine, Haloperidol and Pimozide. However, none of these agents have rendered satisfactory results. The present inventor received U.S. Pat. No. 4,161,382, herein incorporated by reference, for the treatment of schizophrenic patients with AMPT and a urine alkalinizer. Although generally satisfactory, there was still a need for an improved treatment technique.

When treating humans with paranoid schizophrenia and acute mania, exclusively with Haloperidol, and performing daily urinary studies to measure dopamine metabolites, the present inventor found that the metabolites increased gradually and quantitatively in urine while Haloperidol was administered, and that the symptoms of mania, lasting many days or even weeks, started to decrease just when the patient started to present, clinically, symptoms of depression. The urinary studies were done for a period of 6 weeks in eight patients chosen for the study (three manic patients and five schizophrenics of the paranoid type). For three of the paranoid and two of the manic patients, after a month of treatment with heavy doses of Haloperidol, up to 60 mg a day, urinary metabolites still increased and their clinical symptoms were not totally controlled. These five patients needed treatment other than Haloperidol to control the symptoms of their illness. The patients received Akineton to prevent extra-pyramidal side effects, but for three of the patients where Akineton was discontinued from day 8 to day 15, the dopamine metabolites continued increasing as in the cases where Akineton had been given. Therefore, it was concluded that Akineton was not related to the increase in dopamine metabolites.

In order to explain the above findings, the present inventor hypothesized that by blocking the dopamine post-synaptic receptor with Haloperidol, synthesis and release of dopamine increased in the synaptic terminal to overcome the blockade produced by Haloperidol. Hypothetically, the same would occur by using any neuroleptic.

The present inventor hypothesized that the use of AMPT (a synthetic amino-acid that regulates the dopamine and norepinephrine synthesis through its action on the regulatory enzyme tyrosine-hidroxilase) would decrease dopamine synthesis, through the feed-back mechanism of the dopamine autoreceptor. The treatment of humans with the combination of AMPT and Haloperidol resulted in a positive and quick improvement of the acute symptoms in paranoid schizophrenia and manic patients; furthermore, the manifestation of such improvement occurred significantly faster than when using AMPT alone.

Attempts to treat the general spectrum of schizophrenia, other than the paranoid type, have been based on the inventor's belief, that in other types of schizophrenia, dopamine and norepinephrine may intervene as fundamental neurotransmitters. As a consequence the present inventor used AMPT to treat the schizo-affective type, based on the fact that one of the metabolites of AMPT, the alpha-methyl-norepinephrine, has a major affinity for the norepinephrine-receptor rather than the biological metabolite of norepinephrine.

Once armed with a pharmacological tool to regulate the synthesis of dopamine and norepinephrine, the present inventor utilized the methodology described herein to treat schizophrenic patients, in view of the role of said neurotransmitters on the bio-pathology of schizophrenia. In accordance with the present invention, AMPT can be used safely, when administered with a urine alkalinizer, in combination with Haloperidol, for the treatment and controlling of schizophrenia and mania in a quick and effective fashion.

Since the present inventor had obtained such remarkable results for treating schizophrenic patients using the combination of Haloperidol, AMPT, and a urine alkalinizer, it was imperative to attempt the use of this combination for treating manic patients. Both conditions, schizophrenia and manic-depressive psychosis, are extremely close, to the point of being difficult at times to distinguish one from the other. Furthermore, the treatment of both is very similar. In the manic phase of manic-depressive illnesses the role of the neurotransmitter dopamine is recognized as the fundamental one. The symptoms of the manic phase are similar and almost undistinguishable from those of cocaine and amphetamine psychosis, where the acute hallucinatory and delusional symptoms are related to an increased release of dopamine. Therefore the present inventor concluded that if a pharmacological tool (AMPT) was identified that regulates dopamine synthesis, it would be useful to use it in an illness that has alternating phases of symptoms. Such alternating phases would seem to correlate with an excess or decrease in the synthesis and release of dopamine. Surprisingly, AMPT was equally effective in the manic phase to preventing the development of the depressive phase. Moreover, it was demonstrated that AMPT, acting on tyrosine-hydroxilase, is effective in treating illnesses (Mania) where dopamine is increased or decreased in correlation with the clinical phases of manic or depressive symptoms. It is also well known that tyrosine-hydroxylase is the enzyme considered to be the “pace-maker” of catecholamine synthesis and that AMPT is the most appropriate tool to manipulate the function of said enzyme.

The improved results obtained treating narcotic and amphetamine addictions by utilizing Haloperidol, led the present inventor to use AMPT and Haloperidol in the treatment of schizophrenia and mania. It was hypothesized that Haloperidol would produce an almost immediate amelioration of the acute symptoms, by blocking the post-synaptic dopamine receptor. A dose of 10-15 mg t.i.d. of Haloperidol, when added to AMPT, produced an almost total cessation of the acute symptoms in the first 10-14 hours. Such amelioration, when administering AMPT alone, did not occur until 24-30 hours and required higher doses of AMPT than when Haloperidol was added. The case reports of 8-10 below, illustrate the clinical results obtained. Also, Naltrexone was used in treating these mental conditions and clearing the mind. However, when Haloperidol is used with AMPT, Naltrexone is not necessary.

### Use of AMPT, a Urine Alkalinizer and Haloperidol for Treating Tobacco Addiction (Nicotine)

The discovery by the present inventor of the use of AMPT for the treatment of smokers, resulted from treating narcotic addicted patients with AMPT. In nearly 400 treated cases, many of the narcotic addicts reported that they had lost the craving to smoke. Initial research did not pay too much attention to this comment, since it was believed to be a result of a decrease of anxiety-producing factors so common in drug addicts. However, upon further review and specifically questioning the cessation of the craving to smoke for patients receiving AMPT for different conditions, the present inventor found that all smokers had lost their craving to smoke after 2-4 days of treatment with AMPT, and that a significant number of the smokers had quit completely, while others continued smoking to a much lesser degree, although without any craving for it.

While the present inventor was obtaining additional evidence concerning the simultaneous loss of craving for narcotics and smoking, other researchers were establishing the role of dopamine in smoking addiction, to the point that dopamine is considered today to be a fundamental neurotransmitter involved in nicotine addiction. As a result, the present inventor theorized that smoking stemmed from a strong chemical addiction.

In order to prove such a hypothesis, the present inventor resorted again to animal experimentation, using rats and administering nicotine in their drinking water. A cigarette extract was obtained by burning cigarettes and collecting the tar, nicotine, and other products of cigarette combustion by a perforated plastic tube tied to the exhaust of a miniature vacuum cleaner. The air and smoke was passed through a container of water in which the cigarette extract was deposited. This drinking water was subsequently given to the animals. In addition to bottle feeding rats with the cigarette extract water (equivalent to 6 cigarettes per rat per day), the rats were also exposed to cigarette smoke. The rats were exposed for 2 hours at 8 hour intervals, for a total of 6 hours a day, to cigarette smoke in a sealed chamber. After a period of 4 months, 24 animals died of different causes, pneumonia among them, and 66 survived. The remaining 66 were divided in two groups: 35 were treated with AMPT orally, and 31 were kept as controls. The urine was alkalinized in both groups with Polycitra to a pH of about 8. At the end of 4 months, the rats were given the choice to continue drinking the same cigarette extract contaminated water or drink pure drinking water in equal amounts. All animals were given the choice to be in the cigarette smoke filled chamber or to use clean air quarters.

The AMPT treated animals started treatment two days before all animals were given the choice between the cigarette extract drinking water or clean water, or the smoke filled chamber versus a clean air chamber. All animals immediately chose the pure drinking water and the clear air chamber. However, after 8-12 hours, the untreated animals started to drink the cigarette extract drinking water and to step into the smoked filled chamber. This was interpreted as the period of time needed for manifestation of withdrawal symptoms.

However, after 18 hours a clear distinction was observed between treated and untreated animals, the untreated animals all resorted to drinking the cigarette extract water and stepping frequently into the smoke filled chamber. In contrast, the AMPT treated animals chose to continue drinking the pure water and to remain in their cages, stepping only occasionally into the clean air chamber and totally avoiding the smoke filled compartment.

Applying the results of this animal research to human treatment, the present inventor confirmed the same results obtained with treated animals. Furthermore, the present inventor utilized the combination of Haloperidol and AMPT by administering small doses of Haloperidol, 2 mg t.i.d., (6 mgs total per day) for a healthy human, weighing 70 kg, simultaneously with the initiation of treatment with AMPT. This treatment combination was discontinued after 10 days when the Haloperidol was discontinued.

Simultaneous administration of AMPT and Haloperidol for treating smokers, was found to suppress the craving for cigarettes before 24 hours. In contrast, administering only AMPT, the craving did not disappear entirely until 2-3 days of treatment. Therefore, the simultaneous administration of Haloperidol reinforces and improves the positive results obtained with AMPT alone. Generally, the administration of Haloperidol was discontinued after 7-10 days, while AMPT was administered for a period of at least three months in doses of 1 gram t.i.d.

The present inventor formed a hypothesis that the craving and withdrawal of cigarettes, with its own peculiar characteristics, different from narcotics and other addictive states, could be prevented by use of AMPT. In addition to the positive results obtained, the present inventor concluded that tobacco dependence is yet another of the multiple addictions, with all having a common link in which dopamine plays a fundamental role. A dose of Haloperidol of 2 mg t.i.d., in combination with AMPT, resulted in an almost immediate cessation of the craving to smoke, without having the anxiety and other symptoms of withdrawal which are characteristic of stopping cigarette smoking.

In summary, the present invention achieves remarkable results in treating different illnesses by the simultaneous administration of AMPT, Haloperidol, and Naltrexone. Haloperidol was used, instead of other butyrophenones, neuroleptics, or dopamine-receptor blockers, because of having less “autonomic nervous system” side-effects and having major affinity for the dopamine receptor as compared to the neuroleptics. Haloperidol, on its liquid presentation, is odorless and tasteless and as a consequence easy to camouflage.

Although the foregoing description provides guidance for treating the noted conditions, the clinical expertise necessary to satisfactorily treat patients can only be acquired with practice obtained after treating repeated cases of the same condition. The treatment, in most cases, be carried out in an ambulatory fashion and does not need hospitalization. Typically, the treatment regimen requires, at most, the need for a nurse to administer medication at particular times. However, with regard to the addictive states, it may be necessary to deal with dual diagnosis and, after removing the offending drug, then treat the underlying illness, which in many cases was the determining factor to start the use of addictive drugs. Among these, anxiety and depression account for more than 75% of the existing comorbidity.

### Case Report 1 (Heroin)

The patient, male 29 years of age, was born in a middle class family and attended a private school. His father owned an auto-repair shop and his mother stayed at home taking care of the family. He had an older sister and a brother 6 years younger. The brother was addicted to drugs and did not live with the family. At age 15 the patient started to take drugs: LSD, design tablets, speed, etc. Eventually, his habit focused on hashish. One year after he began taking hashish daily, he began, for a brief period of time, taking cocaine. He quit taking cocaine because it caused him to be very talkative, aggressive and paranoid. At age 17, he started to take heroin and in a few weeks he needed it daily. At age 18 he was admitted to an in-patient treatment program for 6 months for heroin dependence. This was followed by one year in a residential drug treatment center, in which he received Naltrexone daily. One week after having been released, he started to consume heroin again. At age 20 he joined the military, but was discharged after three months for drug use. After two additional residential in-treatments, for periods of fourteen months and two and half years, respectively, he was convicted of stealing and sent to jail for 2 years. During all of these events, he never ceased taking heroin. The patient underwent another 3 in-patient treatments during the last 4 years.

The patient, after having been admitted to our clinic, began receiving treatment in accordance with the present invention. AMPT was administered 2 g four times daily and Haloperidol 15 mg four times daily. The urine was properly alkalinized with Polycitra to achieve a pH close to 8. He also received Akineton 2 mgr three times a day. During treatment there was a standing order so that the patient could ask for oral morphine (MST), if he needed it. The patient asked for it only once, five hours after he was started on the treatment. He never had any abstinence symptoms of craving for heroin. Four days after the treatment was initiated he was discharged. Treatment was continued by administering Naltrexone 25 mg daily, AMPT 500 mg t.i.d. Haloperidol 2 mg t.i.d. was ordered to be taken for 7 days. The AMPT was decreased gradually and discontinued totally six months after he had been discharged. A year and a half after he was discharged, the patient is working with his father, without having relapsed back to drugs. The patient has been seen on an out-patient twice a month and both he and his family have confirmed that he has been feeling well and working satisfactorily.

### Case Report 2 (Heroin)

The patient 34 years old and male, became involved with drugs at age 15. The patient had not lived with his family since age 18, and had minimal contact with his parents and brothers. At age 16 he became severely involved in hashish, LSD, speed, and other drugs. He had been rather shy and introverted. He had difficulties making friends and was a poor student. He took heroin for the first time at age 17 and 2 years later he was consuming 1-2 grams daily, stealing money and merchandise in order to support his habit. At age 19 he stayed in a residential treatment center (“El Patriarca”) for 6 months. At age 20 he was hospitalized again in a residential treatment center, where he remained for 2 years. While at the center, he met his first wife, with whom he had 2 children. Two weeks after he was discharged from this second center he was on heroin again and assaulted a store owner with a gun. While in jail for a period of 2 years, he made friends who supplied him with heroin. After being released from jail he lived in England for two years and joined a group of people that consumed drugs heavily. At this time he also consumed cocaine but it made him very nervous. As a result, he used heroin exclusively, which he continued to take in heavy doses. For the last 6 years prior to the present invention treatment he had another 6 hospitalizations and for 2 years he was involved in daily psychotherapy. After release from each hospitalization stay, he would go back to drugs. For a period of 6 months he was maintained on methadone, requiring up to 140 mg a day, but also continued consuming heroin almost daily.

Other than the effects of narcotics upon which the patient was dependent, he was severely anxious, having periods of depression, reporting severe insomnia and irritability. As soon as he was not receiving enough heroin he became extremely aggressive. Before treatment in accordance with the present invention, he was also put on methadone while he underwent physical check-up in order to evaluate his liver condition. He had chronic and present hepatitis, asthma and tuberculosis in the past. It was necessary that the patient was tested for suitability for treatment.

The treatment was initiated with AMPT, 2 g t.i.d. orally and Haloperidol 10 mg t.i.d. The patient was also put on Librium 25 mgr t.i.d. because of his feelings of anxiety. Methadone was discontinued after initiation of the present invention treatment, subject to a standing order that he could receive methadone if he required it. He required it only once 6 hours after initiation of treatment with AMPT and Haloperidol. During the 4 days that the patient was in the hospital, after initiating treatment, he did not have any craving for narcotics. The patient was released on the fifth day after admission at a treatment level of AMPT 500 mg t.i.d., and Haloperidol 2 mg t.i.d. Treatment with antidepressants was established before he left the hospital, together with Naltrexone 25 mg given every other day. The patient was followed as an out patient, on the average of 3 times a month. During 1½ years after release from the hospital, he never returned to drugs. Six months after being discharged he started to work in a family business and was doing satisfactorily.

### Case Report 3 (Cocaine)

The patient, 34 years old, had all his life an inferiority complex. This complex created tremendous difficulties in dealing with his peers, and caused him to feel isolated and moody. He also had multiple phobias that he hid from others, including his spouse. At the age of 17 he started to smoke marijuana, realizing that it was decreasing his anxiety and shyness once he had 2 or 3 joints. The effect was even more remarkable if he had a couple of drinks together with the marijuana. When he was 18 years old, he started to use heroin and cocaine, but confirmed only cocaine as his cousin had died of a heroin overdose. He did not hesitate taking cocaine, since he only needed it on weekends. The use of cocaine became a need, in order to counteract the alcohol which he consumed to calm down and be able to sleep. At the age of 20, he continued his drug use and increased his use of cocaine.

Two years later he needed cocaine daily in order to maintain his mood, not be tired, and be able to carry out his working day. He was going out almost every night with friends who were also taking cocaine. Gradually, he began to consume greater amounts of cocaine, smoking it daily in order to perform his work. Upon discovery by his family of his addiction, he was forced to begin treatment. The patient was taken to a hospital at which he remained for two months. He was treated with “sleep therapy”, and received Narcovenol (a modified barbiturate), and awakened only to eat and use the toilet. After two weeks he was discharged and given Sanaz and Prozac during the day, and Dalmane and Orfidal at night. During his hospitalization, the craving for cocaine never disappeared and one week after release from the hospital he started to take cocaine again. Six months later he was hospitalized again for four weeks. He repeated the same kind of treatment, without benefit, since 5 days after his discharge he started to take cocaine anew. Three months later he began treatment in accordance with the present invention. Prior to treatment, he was consuming an average of 3 grams of cocaine daily, a bottle of whiskey and 3-4 Dalmanes to sleep. The pre-treatment blood work demonstrated a marked elevation of transaminases.

The treatment was initiated with Haloperidol at a dosage of 10 mg four times a day and AMPT 3 g t.i.d., Polycitra was administered to obtain a urine pH of 8. At the same time he received 4 mg of Akineton at night. The patient was discharged after 4 days continuing on Haloperidol at a dosage of 5 mg t.i.d., and AMPT at 1 g four times a day. He also exhibited significant anxiety, multiple phobias and many neurovegetative symptoms. Treatment with Nardil 15 mg three times a day was established after discharge.

Eighteen months after discharge the patient indicated that he felt extremely well, without any desire for cocaine or alcohol, and felt free of anxiety and depression. For the last four months the patient received a treatment regimen of AMPT at 500 mg t.i.d., Haloperidol at 2 mg t.i.d., and Nardil at 15 mg three times a day.

At last report, the patient was doing quite well and he claimed that he had completely lost his craving for cocaine and desire for alcohol. After one year on Nardil at 15 mg three times a day the symptoms of anxiety and depression and his phobias had almost totally disappeared.

### Case Report 4 (Cocaine and Alcohol)

The patient, 44 years old, had consumed alcohol since a very early age. In addition, the patient had, from age 14, used a wide array of hallucinogenic drugs, amphetamines, and LSD. At the age of 18 the patient used cocaine and alcohol exclusively, and in significant amounts. The patient had several detoxification treatments for both cocaine and alcohol, but none had any lasting effect. On three occasions he started to drink and consume cocaine on the same day that he was discharged. Finally, he accepted treatment. He was also somewhat concerned about the potential damage to his liver.

Treatment according to the present invention, was initiated with 2 g AMPT four times a day and Haloperidol in dosages of 10 mg four times a day. Akineton was also administered 2 mg three times a day to prevent extrapyramidal side-effects. On the second day of hospitalization Naltrexone was introduced at 50 mg daily. Twenty-five hours after initiation of the treatment the patient was in a completely different state of mind, without any paranoid symptoms, totally coherent, eutimic and in a very pleasant mood. He was discharged on the fourth day after admission with a treatment regimen of Naltrexone 50 mg daily, AMPT 1.5 g three times a day, and Haloperidol 2.5 mg t.i.d.

The patient has been followed as an out-patient for 18 months without having any craving or desire for alcohol or cocaine since. Six months after treatment the patient indicated that he felt energetic and in a good state of mind.

### Case Report 5 (Alcohol)

The patient, male, 33 years old, married, started drinking at the age of 17. Initially, the drinking was only on weekends. From the age of 24, the drinking was daily and excessive. Typically, the patient would drink two glasses of whiskey when getting up in the morning and consume a couple of bottles during the day. He drank only whiskey. He quit drinking for over one and a half years as a result of pressure from his family. During this period he took daily 500 mg of Disulfiram and 60 drops of Colme, which produced a severe and unpleasant reaction whenever he drank. During those years, however, he felt depressed, often remaining in bed, irritable and very unhappy with his family. As a result, he took an apartment of his own and started drinking immediately again.

Before starting treatment in accordance with the present invention, he drank an average of two bottles of whiskey a day. The patient was admitted to our clinic, where he had a physical examination and hematological tests. Immediately after admission he started to sweat and tremble. The symptoms were suggestive of an impending delirium tremens. The patient was put on Librium 25 mg four times a day and Epilantin. The symptoms remitted in the following 15 hours.

The patient was treated with AMPT 2 g t.i.d. and Neltrexone 50 mg t.i.d., adding Anafranil 25 mg t.i.d. to treat the underlying depression. The patient immediately lost the craving for alcohol and he always felt restful, talkative and complacent five days later he was discharged from the hospital.

The patient continued for one year with Naltrexone, 50 mg daily 6 months decreasing it afterwards to 25 mg a day, and AMPT in doses of 500 mg at breakfast, lunch and dinner, with an alkalinization of the urine in the range of 7.6-8. The patient was seen every 4-6 weeks as an out-patient. Follow up treatment was continued for the next two years without relapsing into alcohol. The phobias were treated with Manerix 150 mg t.i.d. and Huberplex 10 mg t.i.d. The patient's phobias disappeared almost completely and his chronic depression vanished.

### Case Report 6 (Alcohol)

The patient, female, 36 years old, married, had been an excellent student. When the patient started working for the family business, where she had to deal with many people, she felt anxious, insecure and exhausted at the end of the day and experienced difficulties falling asleep. It was then that she started using alcohol. Prior to that, she had only consumed alcohol at family celebrations, at which she excessively drank champagne. In the beginning she started drinking gin and tonic during working hours since she felt that these drinks diminished the anxiety and dryness of the mouth that she constantly experienced. However, returning to her home she needed to consume three or four whiskeys to be able to sleep. During the following three years, whiskey became essential even before leaving the house in the morning. At this point, she was consuming almost a bottle of whiskey every day. During working hours, she would drink 6-8 gin and tonic. She accepted treatment voluntarily.

The patient was admitted into the hospital for a general examination and to avoid complications associated from the sudden withdrawal of alcohol. She was discharged from the hospital three days later to continue treatment at home. She started treatment at the hospital with 50 mg of Naltrexone daily, 2 mg of Haliperidol t.i.d., and 1 g of AMPT t.i.d. The craving for alcohol disappeared and never returned. Because of her phobias, she was also treated with Nardil 15 mg t.i.d. and Librium 10 mg t.i.d. In one of the visits, 4 weeks later, the patient indicated she felt no anxiety, was much more relaxed with people, and experienced a good feeling and mood she did not remember having before. The doses of Naltrexone and AMPT were gradually reduced. After six months Naltrexone was reduced to 25 mg on alternate days and AMPT to 500 mg three times a day. Librium and Nardil were continued at the same doses. After three months of treatment the patient began keeping alcohol in her house for visits by friends and family, without having any desire to drink. Naltrexone and AMPT were suspended after a year of treatment, and two months later Huberplex and Nardelzine were suspended as well.

The patient did not feel compelled to consume alcohol again, but six weeks after stopping Nardil started to feel sad, tired and insecure. On her own account, she started to take Nardil and Huberplex. During the following three years the patient had not taken alcohol but whenever the dosage of Nardil was reduced to two tablets a day, the reduction was always followed by a reappearance of certain anxiety and symptoms of depression. Therefore, the patient requested the medication be continued continuously. The blood analysis conducted every six months did not demonstrate any alterations of the transminases and no other alterations as a consequence of her previous heavy drinking.

### Case Report 7 (Alcohol)

The patient, 26 years old, single woman, was raised in an upper middle class family, being the youngest of four siblings. As a result of a failed relationship, she started drinking excessively. Although she did not have any preference for any specific drink, she preferred dry sherry and table wine. On some days, she consumed a bottle of sherry and two bottles of wine, often being so intoxicated that she was at times, completely unconscious.

Once she was hospitalized, after physical and analytical examinations, the treatment according to the present invention started with 3 mg t.i.d. of Haliperidol, 50 mg of Naltrexone and 2 g of AMPT t.i.d. At the same time, Epanutin 100 g was administered four times daily and Librium 25 mg three times a day, to avoid the complications that could arise as a result of abruptly stopping the intake of alcohol. After 7 days in the hospital, the patient was dismissed with the mentioned doses of Naltrexone and AMPT, without any medication other than Halcion 0.125 mg at bedtime, when needed. The patient was seen at periods of 2-4 weeks over a period of 14 months during which the dosages of Naltrexone were reduced to 25 mg daily and the AMPT to 500 mg three times a day to be discontinued after a year on treatment.

During a period of 2½ years following treatment, the patient did not consume alcohol.

### Case Report 8 (Manic Depressive Psychosis)

The patient, male, 39 years old, had been diagnosed 15 years earlier as manic depressive and 3 years ago as schizophrenic, paranoid type, with mystical delusions. For the last 15 years he has had periods of euphoria and depression. The phases of euphoria would follow with phases of depression during which he was unable to get out of bed and did not even have the strength to maintain his personal hygiene.

When he was hospitalized and began receiving treatment in accordance with the present invention, he was in a manic state voicing many delusional ideas. He was started immediately on 10 mg of Haloperidol every eight hours and 2 g of AMPT every six hours. The patient went into a very relaxing sleep and woke up after 14 hours without having received the third dose of AMPT, which was due after 12 hours. When the patient woke up he was totally coherent without manifesting any paranoid ideology and was not in mania phase anymore. The dose of Haloperidol was reduced to 5 mg four times a day and the dose of AMPT to 1.5 mg four times a day.

On the second day of hospitalization the Haloperidol was reduced to 5 mg four times a day and it was totally discontinued on the third day, when the patient was discharged to go home on AMPT 1.5 g three times a day. After he returned home, he never manifested any paranoid behavior, aggressivity or any pathological symptoms. After six months, the AMPT was decreased to 5 g t.i.d., as a maintenance dose. The patient did not have any more phases of mania or depression and has never mentioned his mystical delusions again. He was continued on AMPT 500 mg t.i.d. and Akineton 2 mg for the last 2 years without any symptoms of illness. Routine urine analysis and blood tests every 6-8 weeks did not reveal any abnormalities.

### Case Report 9 (Manic Depressive Psychosis)

The patient, male, 28 years old, started to have hallucinations and delusions. After one month his manic behavior had changed into depressive phase and he started to drink heavily. After returning home, he was treated in a regional hospital and diagnosed as schizophrenic, paranoid type. He was treated with pimozide and different neuroleptics for a year and a half, until the patient discontinued all medication on his own, without the knowledge of his psychiatrist. After one month he began experiencing delusions.

Then, treatment in accordance with the present invention was initiated. It was obvious that he was in a manic phase. He had not been able to sleep for more than 2-3 hours in the previous week but he was not complaining of any tiredness or lack of energy. Once he was hospitalized, he was immediately administered 10 mg of Haloperidol every eight hours and 3 g AMPT every six hours. Before the third dose of AMPT was given, the patient woke up very relaxed, totally coherent, without manifesting any delusions or reaffirming the ones he had twelve hours before. The dose of Haloperidol was reduced to 5 mg three times a day and AMPT 1.5 g four times a day. That night, the patient was able to sleep, without any other medication and uninterrupted for 8 hours, and when he woke up the following morning, he was in total eutimia, without any delusions and not presenting any signs of depression.

He was discharged from the hospital 46 hours after admission and prescribed 500 mg of AMPT, three times a day and 2 mg three times a day of Haloperidol. After being maintained on this medication for 20 months, an attempt was made to discontinue the medication. However, ten days later he started experiencing delusions and to voice the same paranoid ideas he had before. These symptoms were controlled with 15 mg of Haloperidol at eight hour intervals and increasing the AMPT to 3 g three times a day. After 12 hours from initiation of this treatment, his hallucinations were under control, not voicing them any more, and not presenting any signs of mania. After this relapse, no attempt has been made to discontinue the use of AMPT maintained at a dose of 250 mg t.i.d. The patient has received Akineton 4 mg h.s., to prevent the development of extrapyramidal symptoms.

### Case Report 10 (Schizophrenia)

The patient, male, 39 years old, was diagnosed as schizophrenic when he was 18 years old: he was treated as such by different psychiatrists and with all types of medication, including ECT. During one treatment regimen, he was treated with Eskazine, Haloperidol and a long-acting intramuscular medication (Prolixin). His hallucinations and delusions were controlled but he was very reluctant to continue taking medication. After this treatment, without a complete recovery or able to do any continued work, he was admitted to us in a delusional state again.

In accordance with the present invention, he was administered Haloperidol 10 mg t.i.d. and AMPT 3 g t.i.d. After 24 hours he was without hallucinations, delusions or any abnormal thinking. He could remember all the “imaginations” he had had through the years and discarded them as being nonsense, although admitting that at the time he believed what he said and could not avoid those thoughts. After 6 days of hospitalization he was discharged on Haloperidol 2.5 mg t.i.d. and AMPT 1.5 g t.i.d. The only other medication given was Akinenton 5 mg h.s. The patient was been maintained on the same doses as the day he was discharged during 8 months. Afterwards, he has been seen every 3-4 weeks as out-patient for 1 year, without having any set-backs.

### Case Report 11 (Schizophrenia)

The patient, 36 years old, single woman, had completed secondary education and started experiencing various delusions. At the same time she was in a moody spirit having difficulties with sleeping. Moreover, she had many depressions and became unable to continue her daily activities. She had been given many courses of electroshocks as apparently she was not responding well to any pharmacological treatment. By the age of 27 she had been treated by two different psychiatrists with different pharmacological agents to which she responded poorly. She was placed on long-acting neuroleptics Prolixin, Eskazine and Triavil, that produced a moderate improvement, and controlled her delusions and paranoid ideas, but she remained rather withdrawn from people. The patient continued this treatment for 5 years without seeing any psychiatrist and her medication was supervised by the family doctor, without much progress.

Immediately after being admitted to us, she began treatment in accordance with the present invention. After preliminary testing and physical examination, the patient was started on Haloperidol, 10 mg t.i.d. and AMPT 3 gr t.i.d. All previous medication had been discontinued two weeks before so there was not any interference with previous treatments. On the second day of the present invention treatment, the patient started to show a very notable improvement. After 46 hours from the initiation of the treatment the patient was found with a completely normal flow of ideas, by logical and coherent. After 8 months of training she started to work as a secretary for a television outlet. She exhibited the same degree of carefulness and motivation she had prior to the onset of her illness. She had a follow up of 2 years and has maintained Haloperidol 2 mgrs t.i.d. and AMPT 750 mg t.i.d., without any relapse and showing a gradual improvement.

### Case Report 12 (Nicotine Dependence)

The patient 63 years old, male, had been a heavy smoker of cigarettes and cigars all his life. He was active and in good health until 10 years ago, when he had a mild myocardial infarct, most likely as a result of his previous heavy smoking. At one time, he was smoking two and a half to three packs of cigarettes and 4-6 cigars a day. After the infarct, his family pleaded with him to reduce the number of cigarettes and cigars. He found himself, although aware of the complications, unable to smoke less cigarettes and he never smoked less than 2 packs of cigarettes and 3 cigars a day. When he did attempt to cut down to 1½ packs of cigarettes a day he was nervous, irritable and very despondent. In the last four years he visited a treatment center 3 times, at which, he stayed 4 weeks each time. He tried behavior modification treatment, relaxation techniques, acupuncture, etc., and was able to reduce the number of cigarettes. However, after going back home and into his job he found that he could not stay without cigarettes and gradually started to increase them to two packs a day, although having quit the additional cigars.

When the patient began receiving treatment in accordance with the present invention, he complained of severe insomnia for which he had previously taken sleep medication for the last 10 years and used different benzodiazepines and sedatives. After a complete physical check up, that did not show severe abnormalities to contraindicate treatment, he was started on AMPT 2 g t.i.d. and Haloperidol 3 mg t.i.d. He was concurrently administered Librium 10 mg t.i.d. and the Dalmane and Orfidal at bedtime, that he had received for years. The patient was also administered Akineton 2 mg t.i.d.

Twenty four hours after receiving treatment in accordance with the present invention he had only 8 cigarettes and he reported no need or craving to smoke. On the third day he attempted to smoke a cigarette and found it distasteful and felt as if he would need to force himself to continue. On the fourth day after initiation of treatment, the patient decided to go home. When he returned after 2 weeks, he reported having quit smoking and so has continued to the present (10 months after starting treatment).

### Case Report 13 (Nicotine Dependence)

The patient, a 33 years old, single female, was an effective executive who worked and traveled frequently. She was a brilliant student and successful publisher, and started to smoke when she was 15-16 years old. At age 28 she was traveling all over the world, very successful at her business, but feeling always anxious, and with bouts of depression.

During this period, she smoked 4-6 packs of cigarettes, conscious of how bad it was and the need to quit, but unable to do so without help. She made attempts to quit, each with different treatments. In no instance was she able to smoke less than 2 packs a day.

During her initial evaluation, she talked excessively and in 75 minutes had smoked 36 cigarettes. After initiating treatment with 2 g of AMPT t.i.d. and Haloperidol 6 mg t.i.d. she smoked 18 cigarettes in 8 hours before going to bed. However, on the second day she woke up and had no desire or need to smoke. She spent the entire second day without smoking any cigarettes and so she continued on the third and fourth day. On day 5, the dosage of AMPT was decreased to 1.5 g t.i.d. and the dosage of Haloperidol to 5 mg t.i.d., without having any desire or need to smoke. After 10 days of initiating treatment, AMPT was decreased to I g t.i.d. and Haloperidol was discontinued gradually in the next two days. After 3 months AMPT was reduced to 500 mg t.i.d. without any relapse into smoking. After 10 months from completion of the treatment, the patient continued to refrain from smoking.

Underlying depressions became obvious from the first day. She was administered Librium 10 mg t.i.d. and Anafranil 25 mg t.i.d., with which she had previously exhibited a satisfactory response and reported to feel almost totally free of depression one month after being on the antidepressant medication.

### Case Report 14 (Marijuana Dependence)

The patient, male, 44 years of age, was a chronic marijuana smoker, starting it when, at age 20, he enrolled into the Spanish Legion and was assigned to the north of Africa (Melilla). Since then, he has not stopped smoking 10-15 joints of good marijuana per day, never hashish. He has also drank alcohol heavily, usually cheap grape wine. However, because of serious alterations of liver enzymes, he quit alcohol at age 32 with some relapses thereafter. He quit completely four years before coming to us and continued attending Alcoholic's Anonymous meetings.

The marijuana caused him a lot of trouble because of euphorias and psychotic breaks that ended 3 of his 4 marriages. He also noticed that it impaired his memory greatly, but any attempt to quit ended in failure even after repeated treatments. He also took amphetamines, L.S.D., sniffed glue, and smoked cigarettes as a youngster, but after that, he confined himself to marijuana and alcohol. Admitted to us to be treated with the present invention, he had a physical and psychiatric evaluation that revealed heart and chest abnormalities with severe impairment of memory and concentration and decreased retention and calculo abilities. All of the above made us consider the existence of an organism brain syndrome, with underlying depression and marijuana dependence.

Treatment according to the present invention was initiated with AMPT 3 gr t.i.d., Haloperidol 10 mg t.i.d., and Naltrexone 50 mg daily, Akineton 2 mg t.i.d. was also given to prevent extrapyramidal side-effects. The medication was decreased gradually. After 8 days of hospitalization, the patient was discharged on AMPT 1.5 gr t.i.d., Haloperidol 2 mg t.i.d., Naltrexone 50 mg q.i.d., and Akineton 2 mg b.i.d.

Seen on an out-patient basis twice a week, he reports no craving or desire for marijuana at all. He has not smoked marijuana since treatment was initiated, and has not had any abstinence symptoms. Treatment with antidepressants was started one month after discharge from the hospital

### Case Report 15 (Marijuana Dependence)

The patient, male, 24 years of age, had started to smoke marijuana at age 16, together with alcohol and amphetamines, for which he was treated at ages 18 and 20, giving up alcohol and street drugs, except for marijuana which he considered harmless, although he noticed after intensive use, that it was making him extremely relaxed and with no motivation, and when smoking hashish euphoric and paranoid. He also believed that marijuana caused him to abandon his studies and lose any jobs he had.

Admitted to us to be treated with the present invention, he received AMPT 2 g q.i.d., Haloperidol 8 mg t.i.d., Akineton 2 mg t.i.d. and Naltrexone 50 mg q.d. After 5 days of hospitalization, he was discharged on AMPT 1.5 g t.i.d., Haloperidol 3 mg t.i.d., Akineton 2 mg b.i.d., and Naltrexone to be continued at 50 mg q.d.

The patient reported no need for marijuana from the initiation of treatment and having no abstinence manifestations. After discharge, he continued to be seen on an out-patient basis twice a week without relapse into his habit.

As previously noted, the present invention relates to the discovery that administering a combination of ANPT in conjunction with Haloperidol is superior over the administration of solely AMPT. Case reports 16-19 set forth below provide examples of such treatment practices.

### Case Report 16 (AMPT alone)

The patient, a 41-year-old married male, was the youngest of his family and has two siblings. At the age of 25, the patient was accepted in our hospital and later discharged the following year.

At the age of 14, he started to take hallucinogens and inhale glue, petroleum products and any other substance that he thought could provide new sensations for him. Along with his friends, he began to smoke marijuana and later began to consume designer drugs, as well as great quantities of metaqualona with alcohol. When he was 17, after having taken cocaine a few times, he also started to take heroin, smoking it at first, and, after one week, injecting it intravenously, a habit he could no longer break. He stopped consuming other drugs but he kept injecting himself with heroin, smoking marijuana and hash. At the age of 19 he was hospitalized in a rehabilitation center specializing in drug treatment, although two weeks before being discharged, he again started injecting himself with heroin and consuming cannabis.

The patient returned to heroin and hash consumption.

After two brief hospitalizations with follow-ups, without getting off heroin, the patient was suggested to visit our clinic for treatment. Here, a problem was not only detected with heroin usage, but also an anxiety process-depression, with great axiety, autonomic symptoms and phobias. Those phobias had already appeared before he started taking drugs. When he was 9 or 10 years old, there were occasions when he was not able to attend school due to colitis attacks that, however, would never appear on the weekends or during holidays. Therefore, in conjunction with the treatment for heroin, it was necessary to start treatment for depression with a follow up, which resulted in breaking the addiction to heroin. He was discharged after 65 days in the hospital, free of drugs and his associated psychiatric condition was also treated. The patient is now a successful businessman, as well as an excellent father and husband.

### Case Report 17 (AMPT alone)

This female patient, 28 years old at the time she began treatment, was the only daughter and eldest child of a family with four children. She had studied in Switzerland, had an excellent education, the best grades and numerous academic successes.

During her first year at college, she began to exhibit rather odd behavior. The patient's strange behavior was reported to her parents by college personnel and also by some fellow students who had noticed a change from the peaceful person she was at the beginning of the term to the aggressive and protesting character she had become. Her parents immediately sought psychiatric assistance for her. The diagnosis and treatment were for manic phase in a manic-depressive condition like the one suffered by her mother and a maternal uncle. When she was discharged from a psychiatric center, she returned to her home where she continued with the treatment and had continuing euphoric and depressive stages throughout the years, which prevented her from proceeding with her studies in architecture.

When she was 23, she began a relationship with a drug user. Under her companion's influence, the patient began consuming designer drugs, drinking alcohol in excess and later resorting daily to cocaine and heroin while still consuming cannabis, the drug she had started with. Between the ages of 24 and 27, she was hospitalized three times for extended periods, interned in a center for drug addiction treatment, but every time she immediately went back to drug consumption after being discharged. Having had a “fit of madness” after a high intake of cocaine, she did stop taking it for two years before her admission to our service. At that time, she was only having intravenous heroin and smoking hashish, however, in increasingly higher quantities of both.

The patient was admitted in our service for treating her addiction to heroin which she was consuming in high quantities. As far as she was concerned, hashish was a harmless drug that she did not intend to abandon, but our efforts were aimed at her abandoning it, too. She was treated with AMPT and put on methadone in order to gradually abolish her craving, with no abstinence symptoms appearing. Aware that the patient had a mixed type bipolar depression, when depressive symptoms appeared, a treatment was started for her bipolar depression, reinstating appropriate medications. She was discharged after 78 days of hospitalization, free of drugs and with her psychiatric illness well compensated. The patient was followed up by us on an external basis, and she had not taken any drugs during the 14 months since she was discharged.

### Case Report 18 (AMPT and Haloperidol)

This female patient, 20 years old, was referred to us for heroin treatment immediately after her parents became aware of her problem. The patient had been smoking marijuana since she was 17.

At 18, the patient had finished high school and began to study at a university. She began to socialize with a group of drug users. They induced the patient to take cocaine, hashish and heroin. Never avoiding the consumption of joints and alcohol, in one year she increased the dose of heroin, which she took intravenously, to one gram a day.

The patient was then admitted to our service.

Blood and urine analysis made upon admission showed no contraindication to the treatment and she was immediately put on specific medication. Chromatography was positive for opiates, cannabis and cocaine, as could be expected on the basis of information provided by the patient. After starting the treatment she was informed, as usual with patients of this kind, that there was a standing order for a narcotic to be taken orally should she feel any craving or abstinence symptoms; but the patient never required it. Chromatographies were negative one week later and the patient was discharged 10 days after admission, free of craving and without ever having abstinence symptoms. When intake of narcotic and stimulant drugs was stopped, the patient started to show symptoms of depression, and anti-depression treatment was established five days after admission. After the patient's bothersome orthostatic hypotension was corrected, she was discharged to go home.

The patient was seen with different intervals, as an outpatient, without ever requiring hospitalization and having never relapsed into drug consumption. When she was seen two weeks after discharge, she reported that she was in the best of spirits and had not felt so well for many years. She intended to resume her studies in January the next year. She continued as an outpatient with visits gradually less frequently. A great number of chromatographies and tests were made, always unexpectedly, or when her parents suspected she may have returned to her previous practices. The patient gave up smoking marijuana, never took heroin or cocaine again, and only drank alcohol in moderation.

### Case Report 19 (AMPT and Haloperidol)

The patient, married, 49 years old, had been formerly treated by us and discharged four weeks after his initial admission, free of craving for cocaine and with no need or craving for alcohol. During the noted hospitalization, he underwent a rhinoplasty which apparently, was not very successful, and he suffered a dilatation of the urethra as a consequence of several venereal infections that had taken place in his youth. A treatment was also established for his depression, which he had had for many years, with crisis of panic and specific phobias. After being discharged, he was seen as an external patient with a visiting frequency increasingly more extended at intervals of 2-3 weeks. The patient continued in good condition.

Before this first hospitalization with us he had been under many treatments, both as an external and internal patient, coping with only two of them. After 4-6 months, he was discharged with his doctor's consent. In most other cases, always in private centers, he used to leave after a few days, driven by his desire for cocaine, a craving that never disappeared until he had his first treatment with us.

The patient had been extremely introverted, with many fears of a phobic nature, which made him feel miserable.

Around the age of 16, he began going out with his friends. In their company, he began smoking marijuana and hashish and taking amphetamines and L. S.D. With the passing of years, he became more inclined towards cocaine and alcohol. The patient never tried heroin which the group usually took intravenously. He relapsed after his initial treatment (previously noted), after which he was feeling well with respect to his depression, free of most of his phobias and happy for the first time with his work and his family of three children.

The patient went on taking cocaine and alcohol in doses increasingly higher, reaching in three months almost the same daily doses he used to have before the former treatment, simultaneously or drinking whisky by the gallon after each streak of cocaine.

The patient was admitted to our treatment on and discharged 9 days later to be seen as an out-patient as required.

The follow-up during 14 months did not reveal any setback in his condition.

### Results of This Invention with Reference to the Previous Ones

By utilizing Haloperidol in combination with AMPT, the results of previous treatment with AMPT and Polycitra are significantly improved, in terms of reducing the treatment period to about 20% of the time previously required. Moreover, hospitalization is unnecessary in most cases. Furthermore, a variety of new conditions (alcohol, cocaine, marijuana) may now be treated by use of the present invention.

The present invention could also include the potential use of other agents instead of AMPT and Haloperidol. For example, instead of utilizing AMPT, one or more isomers, analogues, or esters of AMPT could be employed and quite often we have used the levogyro form of AMPT, but the racemic mixture is less expensive to synthesize and accomplishes the same results. Additionally, the treatment regimens may include other agents besides those described herein. Accordingly, while the preferred embodiments of this invention have been described above, it will be apparent to those skilled in the art that various changes and modifications may be made without departing from the body of this invention. Therefore, the claims, as set forth below, are intended to encompass all such changes and modifications that fall within the spirit and scope of the present invention.

The preferred embodiments described herein provide numerous advantages over known prior art treatment techniques. A wider array of conditions may now be treated. The preferred embodiment treatment techniques are generally accomplished in a shorter period of time and with greater effectiveness. The treatment techniques reduce cost and expense to the patient and often eliminate hospital stay. As a result, patients can typically be treated at their home and in familiar surroundings.

Accordingly, while the preferred embodiments of this invention, at present, have been described, it will be apparent to those skilled in the art that various changes and modifications may be made without departing from the invention. And, therefore, the claims, as set forth below, are intended to encompass all such changes and modifications that fall within the spirit and scope of the present invention.

Having thus described the preferred embodiments, I claim:

1. A pharmaceutical composition comprising:
an effective dosage amount of alpha-methyl-para-tyrosine; and
an effective dosage amount of 4-[4-(p-chlorophenyl)-4-hydroxy-piperidino]-4′-fluorobutyrophenone.

2. The composition of claim 1 further comprising:
an effective amount of a urine alkalinizer.

3. The composition of claim 1 wherein said effective dosage amount of alpha-methyl-para-tyrosine ranges from about 1 mg to about 200 mg per kg of body weight per day.

4. The composition of claim 3 wherein said effective dosage amount of alpha-methyl-para-tyrosine ranges from about 15 mg to about 50 mg per kg of body weight per day.

5. The composition of claim 1 wherein said effective dosage amount of 4-[4-(p-chlorophenyl)-4-hydroxy-piperidino]-4′-fluorobutyrophenone ranges from about 0.015 mg to about 1.0 mg per kg of body weight per day.

6. The composition of claim 5 wherein said effective dosage amount of 4-[4-(p-chlorophenyl)4-hydroxy-piperidino]-4′-fluorobutyrophenone ranges from about 0.05 mg to about 0.80 mg per kg of body weight per day.

7. The composition of claim 1 further comprising:
an effective dosage amount of Naltrexone.

8. A pharmaceutical composition comprising: an effective dosage amount of alpha methyl paratyrosine; and an effective dosage amount of Naltrexone.

9. A method for treating at least one of (i) addiction to heroin, narcotics, cocaine, amphetamines, alcohol, nicotine or marijuana; and (ii) schizophrenia or manic depressive psychosis, said method comprising:
administering an effective dosage of alpha-methyl-para-tyrosine to a patient in need of such treatment; and
administering an effective dosage amount of 4-[4-(p-chlorophenyl)-4-hydroxy-piperidino]-4′-fluorobutyrophenone to said patient.

10. The method of claim 9 wherein said effective dosage amount of alpha-methyl-para-tyrosine ranges from about 1 mg to about 200 mg per kg of body weight per day.

11. The method of claim 9 wherein said effective dosage amount of 4-[4-(p-chlorophenyl)-4-hydroxy-piperidino]-4′-fluorobutyrophenone ranges from about 50 mg to about 185 mg per kg of body weight per day.

12. The method of claim 9 wherein said effective dosage amount of 4-[4-(p-chlorophenyl)-4-hydroxy-piperidino]-4′-fluorobutyrophenone ranges from about 0.015 mg to about 1.0 mg per kg of body weight per day.

13. The method of claim 9 wherein said effective dosage amount of 4-[4-(4-(p-chlorophenyl)-4-hydroxy-piperidino]-4′-fluorobutyrophenone ranges from about 0.05 mg to about 0.80 mg per kg of body weight per day.

14. The method of claim 9 further comprising:
administering an effective dosage amount of Naltrexone.

15. A method for treating alcoholism or marijuana addiction comprising:
administering an effective dosage amount of alpha-methyl-para-tyrosine; and 4-[4-(4-(p-chlorophenyl)-4-hydroxy-piperidino]-4′-fluorobutyrophenone.

16. A method for treating alcoholism comprising:
administering an effective dosage amount of alpha-methyl-para-tyrosine; and
administering an effective dosage amount of Naltrexone.

17. A pharmaceutical composition consisting essentially of:
an effective dosage amount in the range from about 1 mg to about 200 mg per kg of body weight per day of alpha-methyl-para-tyrosine; and
an effective dosage amount in the range from about 0.015 mg to about 1.0 mg per kg of body weight per day of 4-[4-(p-chlorophenyl)-4-hydroxy-piperidino]-4′-fluorobutyrophenone.

18. The composition of claim 17 wherein said effective dosage amount of alpha-methyl-para-tyrosine ranges from about 15 mg to about 50 mg per kg of body weight per day.

19. The composition of claim 17 wherein said effective dosage amount of 4-[4-(p-chlorophenyl)-4-hydroxy-piperidino]-4′-fluorobutyrophenone ranges from about 0.05 mg to about 0.80 mg per kg of body weight per day.
